(19) United States
(12) Patent Application Publication
(10) Pub. No.: US 2022/0023196 A1
(43) Pub. Date: Jan. 27, 2022

(54) COSMETIC COMPOSITION FOR SKIN IMPROVEMENT COMPRISING GREEN BARLEY EXTRACT

(71) Applicants: AMI COSMETIC CO., LTD., Seoul (KR); DAEBONG LS CO., LTD., Incheon (KR)

(72) Inventors: Kyung Rok LEE, Seoul (KR); Kyung Min KIM, Jeju-si (KR); Su Yeong KIM, Jeju-si (KR); Ji Mi KANG, Jeju-si (KR); So Young SHIN, Jeju-si (KR); Ji Hun PARK, Jeju-si (KR); Jin Oh PARK, Seoul (KR); Ji Won LEE, Seoul (KR); Hye Ja LEE, Seogwipo-si (KR); Ji Hye KIM, Seogwipo-si (KR); Ho Young KIM, Incheon (KR)

(21) Appl. No.: 16/988,641

(22) Filed: Aug. 8, 2020

(30) Foreign Application Priority Data
Jul. 24, 2020 (KR) 10-2020-0092306

(51) Int. Cl. A61K 8/9794; A61K 8/60; A61K 8/68; A61Q 19/00

(52) U.S. Cl. CPC A61K 8/9794; A61K 8/602; A61Q 19/00; A61Q 19/007; A61Q 19/005; A61K 8/68

(57) ABSTRACT

Provided is a cosmetic composition for skin improvement including a green barley extract.

### CROSS-REFERENCE TO RELATED APPLICATION

This application claims priority to and the benefit of Korean Patent Application No. 10-2020-0092306, filed on Jul. 24, 2020, the disclosure of which is incorporated herein by reference in its entirety.

### BACKGROUND

### 1. Field of the Invention

The present invention relates to a cosmetic composition for skin improvement including a green barley extract.

### 2. Discussion of Related Art

The skin, which is an outer covering layer of the body, performs various physiological functions for protecting the body from various environmental factors such as external stimuli, damage, and dryness, and thereby protects and helps to regulate internal organs and miscellaneous internal body parts.

However, as the body ages, since the physiological function of the body is lowered, the secretion of various hormones regulating metabolism decreases, and immune cell functions and cell activity are lowered. In addition, when the skin is repeatedly exposed to light or heat, the appearance or function of the skin changes, causing the skin to be exposed more to free radicals, harmful reactive oxygen species, and the like. In this case, due to the excessive physical stimuli, chemical stimuli, stress, and the like which promote the deterioration of normal functions of the skin, the formation of blemishes, freckles, and the like caused by melanin deposition, and skin aging, the skin is damaged.

In order to keep the skin healthy and beautiful by preventing skin aging and damage, efforts are being made to keep the skin healthy by promoting skin metabolism by maintaining the skin's inherent functions and activating skin cells, and to this end, various physiologically active materials obtained from animals, plants, microorganisms, and the like have been used in cosmetics, and cosmetics, external preparations for the skin, and the like have been developed.

However, these materials have safety issues, such as causing irritation, erythema, redness, and the like, when applied to the skin and thus have limited usage, or they have little effect on the skin and thus are not expected to have a substantial skin function improving effect. Therefore, there is a need to develop a new external preparation composition for the skin that is safer for the body than existing external preparation compositions for the skin and has high effectiveness.

### RELATED-ART DOCUMENT

### Patent Document

(Patent Document 1) Korea Laid-Open Patent Application No. 10-2019-0088920

### SUMMARY OF THE INVENTION

The present invention is directed to providing a cosmetic composition for skin barrier strengthening or skin moisturization including a green barley extract.

One aspect of the present invention provides a cosmetic composition for skin barrier strengthening or skin moisturization including a green barley extract.

In the present invention, “green barley”, which is a type of barley (Hordeum vulgare L.), refers to greenish unripe barley belonging to the family of monocotyledonous flowering plants in the order Poales and is called Cheongmaek in Korea. Green barley is known as a repository of nutrients due to it containing various vitamins, minerals, amino acids, and the like necessary for the human body, and the results of previous studies show that green barley contains a large amount of components related to stamina, such as superoxide dismutase (SOD), hexacosanol, octacosanol, and policosanol, as well as 18 amino acids, 15 vitamins, 8 minerals, and chlorophyll. Specifically, in the present invention, “green barley” may refer to the young-leaf barley grown on Jeju Island, Korea.

In the present invention, “skin barrier” is a part which, in the skin structure consisting of the epidermis exposed on the outside and the dermis located beneath the epidermis, corresponds to the stratum corneum located at the outermost part of the epidermis. The role of the skin barrier is to prevent moisture from escaping while preventing external harmful elements from entering the skin. In the present invention, “skin barrier strengthening” refers to preventing the skin from becoming vulnerable to external stimuli, easily losing moisture and becoming dry, and becoming sensitized due to the weakening of the skin barrier or refers to improving such problems.

In the present invention, “skin moisturization” includes both supplying moisture to the skin to replenish the moisture of the skin and protecting the skin from losing moisture. In general, the percentage of moisture in the skin, which is about 70% in the dermis, decreases toward the epidermis and is about 10 to 30% in the stratum corneum. When the percentage of moisture in the cell layers decreases to less than 10%, since the skin becomes rough and the skin's ability to protect the body weakens, aging occurs. Therefore, hydration and moisturization are important for skin protection.

In the present invention, in order to obtain green barley that is safe for the human body and eco-friendly as a material for functional cosmetics, it was intended to cultivate green barley without using pesticides and while blocking environmental hazards. To this end, a sprout cultivator was developed, and green barley was cultivated under optimum conditions where external influences had been minimized to enable the cultivation of functional crops of consistent quality throughout the year and maximize components having functions such as skin moisturization and skin barrier strengthening. Specifically, the green barley may be grown for two to six weeks in the sprout cultivator.

In one embodiment of the present invention, green barley is cultivated using a sprout cultivator 100 including: a plurality of trays 10 having small holes for allowing the roots of sprouts to descend; cultivation plates 20 provided under each of the trays 10 and including waterways for circulating water or a cultivate solution; a stacking rack 30 for enabling a plurality of the cultivation plates 20 to be stacked in multiple layers; a cultivation solution circulator 40 for circulating water or a cultivation solution through the cultivation plates 20; a water tank 50 for storing the water or cultivation solution to be supplied to the cultivation plates 20 by the cultivation solution circulator 40; a first transfer pipe 60 for transferring the water or cultivation solution from the water tank 50 to the cultivation plates 20; and a second transfer pipe 70 for transferring the water or cultivation solution from the cultivation plates 20 to the water tank 50 (FIG. 1).

The sprout cultivator having the above-described structure has the advantages that the circulated water or cultivation solution can be reused, and in particular, it is possible to cultivate crops regardless of the season, and in particular, it is possible to cultivate crops in a way that is safe for the human body and eco-friendly, by not using pesticides and blocking environmental hazards.

In addition, since the optimum conditions for growing crops are continuously provided, it is possible to grow crops faster than growing crops in an open field, and since the crops are spatially separated from the nearest contaminated external environment, eco-friendly cultivation is enabled.

In the present invention, “extract” includes the green barley extract itself and all types of formulations formed using the green barley extract, such as extracts obtained by extracting green barley, diluted solutions or concentrates of the extracts, dried products obtained by drying the extracts, preparations or purified products of the extracts, and mixtures thereof.

The extract of the present invention may be extracted from the natural green barley or a hybrid or variety thereof, or may be extracted from plant tissue cultures.

The method of preparing the extract of the present invention is not particularly limited, and the extract may be prepared by a method commonly used in the art. Non-limiting examples of the extraction method include a hot-water extraction method, an ultrasonic extraction method, a filtration method, and a reflux extraction method, which may be used alone or in combination of two or more thereof.

Types of extraction solvent used in the present invention are not particularly limited, and any solvent known in the art may be used. As a non-limiting example, the extraction solvent is one or more selected from the group consisting of water (purified water), a C₁-C₄ anhydrous or lower alcohol, a mixed solvent of the water and the lower alcohol, acetone, 1,3-butylene glycol, ethyl acetate, and chloroform, which may be used alone or in combination of two or more thereof.

Specifically, in the present invention, ethanol may be used as the extraction solvent. More specifically, the green barley may be extracted for one to four hours at a temperature of 50 to 70° C. using a 40 to 60% ethanol after being dried at a temperature of 40 to 70° C.

The green barley extract may contain saponarin represented by Chemical Formula 1.

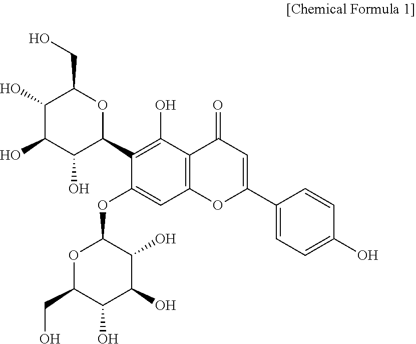 [SMILES: O=c1cc(-c2ccc(O)cc2)oc2cc(O[C@@H]3O[C@H](CO)[C@@H](O)[C@H](O)[C@H]3O)c([C@@H]3O[C@H](CO)[C@@H](O)[C@H](O)[C@H]3O)c(O)c12]

The “saponarin” refers to a type of flavone glucoside derived from green barley and having the chemical name 5-hydroxy-2-(4-hydroxyphenyl)-6-[3,4,5-trihydroxy-6-(hydroxymethyl)oxan-2-yl]-7-[3,4,5-trihydroxy-6-(hydroxymethyl)oxan-2-yl]oxychromen-4-one. The saponarin may be isolated from natural sources such as green barley (e.g., young-leaf green barley), but the present invention is not limited thereto, and any material chemically synthesized by a method known in the art or a commercially available material may be used.

Specifically, the saponarin may be contained at 0.01 to 10% by weight. More specifically, the saponarin may be contained at 30 to 3,000 ppm.

In one embodiment of the present invention, it was confirmed that the saponarin material was contained in the green barley extract, and it was confirmed that the smart-farm green barley extract produced by the method disclosed in FIG. 1 contained a high level of saponarin. (FIGS. 2 to 4A-4C)

In one embodiment of the present invention, it was confirmed that the saponarin significantly increased the expression levels of hyaluronic acid (HA), aquaporin 3 (AQP3), and filaggrin (FLG) (FIGS. 6 and 7A-7B), and it was confirmed that the green barley extract containing the saponarin component was also a material having a remarkable effect on skin moisturization and skin barrier strengthening by increasing the expression levels of all of HA, AQP3, and FLG (FIGS. 8 and 9A-9B).

The green barley extract may be included in an amount of 0.001 to 30% by weight based on the total weight of the composition. When the content of the green barley extract is within the above-described range, there are advantages that excellent skin barrier strengthening and moisturizing effects are exhibited and the formulation of the composition is stabilized.

The composition may further include one or more ceramides selected from the group consisting of ceramide EOP, ceramide NS, ceramide NP, ceramide AS, and ceramide AP.

In the present invention, “ceramide” is a type of sphingolipid-based compound having a structure in which a fatty acid is linked to sphingosine or phytosphingosine. Specifically, the ceramide may be represented by the following Chemical Formula 2, where R represents an alkyl portion of the fatty acid.

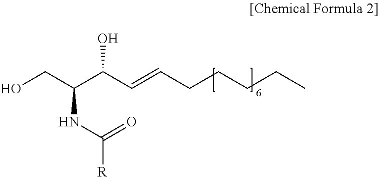

Ceramide is a major lipid component constituting the stratum corneum and plays a critical role in the formation of the skin's moisturizing ability and the occurrence of skin barrier diseases. Ceramide accounts for at least about 40 to 50% of the keratinocyte intercellular lipids constituting the stratum corneum of the skin, is an essential component in the formation of the structure or function of the stratum corneum, and serves as a lipid barrier inhibiting the evaporation of moisture in the stratum corneum and maintains the orderly structure of the stratum corneum.

Ceramide has a skeletal name S, P, or H depending on whether its basic structure is sphingosine, phytosphingosine, or 6-OH-sphingosine, respectively, and may further be classified into EOS (ceramide 1), EOP (ceramide 9), EOH (ceramide 4), NS (ceramide 2), NP (ceramide 3), NH (ceramide 8), AS (ceramide 5), AP (ceramide 6), and AH (ceramide 7) according to the bonding form of a fatty acid.

Ceramides have different polarities, and may be classified into ceramide EOP, ceramide NS, ceramide NP, ceramide AS, and ceramide AP depending on the degree of polarity.

In the case of ceramide EOP, it is known that when production capacity is low, since keratinocytes of the skin cannot sufficiently produce skin lipids, dermatitis such as atopic dermatitis occurs. Therefore, ceramide EOP is mainly used for improving the condition of dry and itchy skin.

In addition, ceramide NP is a complex active material and the most important material among fats and oils contained in the stratum corneum of the skin, and is particularly highly effective for moisturizing the skin. Ceramide NP is mainly known to give shine, gloss, and elasticity to hair and play a key role in strengthening the lipid barrier of the skin and forming a film for protecting the skin against the external environment.

Specifically, the ceramide may be a combination of ceramide EOP, ceramide NS, ceramide NP, ceramide AS, and ceramide AP.

The ceramide EOP, ceramide NS, ceramide NP, ceramide AS, and ceramide AP may be represented by the following Chemical Formulas 3 to 7, respectively.

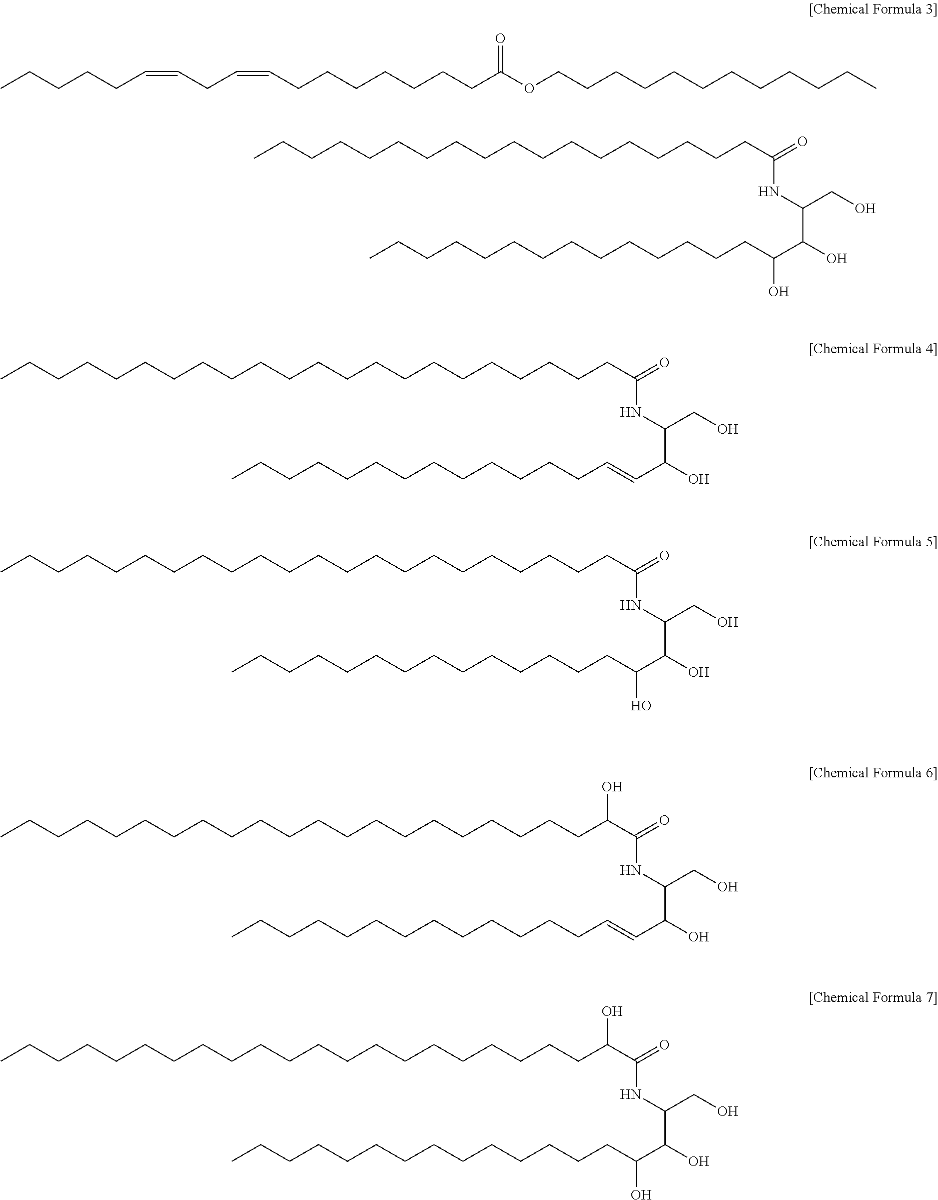

Each of the ceramide EOP, ceramide NS, ceramide NP, ceramide AS, and ceramide AP may be used alone, or a mixture in which two or more of these different types of ceramide are included in a predetermined ratio may be used.

In addition, the ceramide(s) may be used in an amount of 0.01 to 10% by weight based on the total weight of the composition.

In one embodiment of the present invention, it can be seen that from the fact that a mixed composition of green barley extract and ceramide significantly increased the expression level of FLG (Table 5) and the expression levels of HA and AQP3 (Tables 6 and 7), the use of ceramide produces an excellent synergistic effect in regard to strengthening the skin barrier and moisturizing the skin.

The cosmetic composition of the present invention may be prepared into a formulation selected from the group consisting of a solution, an ointment for external use, a cream, a soothing gel, a foam, a nourishing toner, a softening toner, a pack, a softener, a body wash, a milky lotion, a makeup base, an essence, a soap, a liquid cleanser, a bath, a sunscreen cream, a sun oil, a suspension, an emulsion, a paste, a gel, a lotion, a powder, a foam cleanser, an oil, a powder foundation, an emulsion foundation, a wax foundation, a patch, and a spray, and specifically, may be in one or more formulations selected from the group consisting of a soothing gel, a lotion, a cream, a body wash, a foam cleanser, an essence, a powder, a pack, a mask, and a toner, but the present invention is not limited thereto.

The formulation is not irritating and is suitable for the itchy skin caused by skin dryness, so it is usable without limitation even on sensitive skin.

In one embodiment of the present invention, in which soothing gel, lotion, cream, body wash, and foam cleanser formulations including the green barley extract or a combination of the green barley extract and ceramide were prepared and applied to the skin for four weeks to evaluate the ability of the formulations to moisturize the skin and improve itchy skin and the safety of the formulations for sensitive skin, it was confirmed that all the formulations are effective for moisturizing the skin without having side effects (Tables 11 to 16) and are non-irritating products that do not cause irritation even on sensitive skin (Tables 19 to 25).

Furthermore, in one embodiment of the present invention, it was confirmed that the cosmetic composition is also highly effective for improving the itching caused by dry skin and is therefore effective for improving dry skin (FIGS. 11A-11F).

The cosmetic composition of the present invention may further include one or more cosmetically acceptable carriers used in general skin cosmetics, and may suitably include typical ingredients such as an oil, water, a surfactant, a moisturizer, a lower alcohol, a thickener, a chelating agent, a pigment, a preservative, and a fragrance, but the present invention is not limited thereto.

Types of the cosmetically acceptable carrier used in the cosmetic composition of the present invention may vary depending on the formulation of the cosmetic composition.

When the formulation of the present invention is an ointment, a paste, a cream, or a gel, an animal oil, a vegetable oil, a wax, paraffin, starch, tragacanth, a cellulose derivative, polyethylene glycol, silicone, bentonite, silica, talc, zinc oxide, or the like may be used as a carrier component, but the present invention is not limited thereto. The component may be used alone or in combination of two or more thereof.

When the formulation of the present invention is a powder or a spray, lactose, talc, silica, aluminum hydroxide, calcium silicate, a polyamide powder, or the like may be used as a carrier component, and in particular, when the formulation of the present invention is a spray, a propellant such as chlorofluorohydrocarbon, propane/butane, or dimethyl ether may be additionally used, but the present invention is not limited thereto. The component may be used alone or in combination of two or more thereof.

When the formulation of the present invention is a solution or an emulsion, a solution, a solubilizing agent, an emulsifying agent, or the like may be used as a carrier component. For example, water, ethanol, isopropanol, ethyl carbonate, ethyl acetate, benzyl alcohol, benzyl benzoate, propylene glycol, 1,3-butyl glycol oil, or the like may be used as a carrier component, and in particular, cottonseed oil, peanut oil, corn seed oil, olive oil, castor oil, sesame oil, an aliphatic glycerol ester, polyethylene glycol, or a fatty acid ester of sorbitan may be used as a carrier component, but the present invention is not limited thereto. The component may be used alone or in combination of two or more thereof.

When the formulation of the present invention is a suspension, a liquid diluent such as water, ethanol, or propylene glycol, a suspending agent such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like may be used as a carrier component, but the present invention is not limited thereto. The component may be used alone or in combination of two or more thereof.

The composition of the present invention may be used by a transdermal administration method such as directly applying or spraying on the skin, and the composition of the present invention may be administered through any general route as long as it can reach a target tissue through the route.

The usage amount of the composition of the present invention may be suitably adjusted according to individual differences (e.g., age, the severity of lesions, etc.) or a formulation type, and the composition may be used by way of applying a suitable amount to the skin once to several times a day for one week to several months.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a schematic view of a sprout cultivator for cultivating green barley;

FIG. 2 shows the result of comparing green barley extracts to identify the presence of saponarin;

FIGS. 3A-3B show chromatograms (A: standard, B: green barley extract);

FIGS. 4A-4C show the result of mass spectrometry (MS) (A: saponarin, B: luteolin, C: apigenin);

FIG. 5 shows the cell viability of green barley extract;

FIG. 6 shows the result of measuring the amount of FLG produced by saponarin;

FIGS. 7A-7B show the result of measuring the amounts of skin moisturizing factors produced by saponarin (A: HA production amount, B: AQP3 production amount);

FIG. 8 shows the result of measuring the amount of FLG produced by a green barley extract;

FIGS. 9A-9B show the result of measuring the amounts of skin moisturizing factors produced by a green barley extract (A: HA production amount, B: AQP3 production amount);

FIGS. 10A-10D are photographs showing skin irritation reactions caused by a human patch test (A: level 1 (+), B: level 2 (++), C: level 3 (+++), D: level 4 (++++)); and

FIGS. 11A-11F show the questionnaire evaluation result regarding dryness-induced itching according to types of formulation. (A: an average questionnaire-based evaluation score for itching when a soothing gel formulation was used, B: the degree of improvement in itching by a soothing gel formulation, C: an average questionnaire-based evaluation score for itching when a lotion formulation was used, D: the degree of improvement in itching by a lotion formulation, E: an average questionnaire-based evaluation score for itching when a cream formulation was used, F: the degree of improvement in itching by a cream formulation)

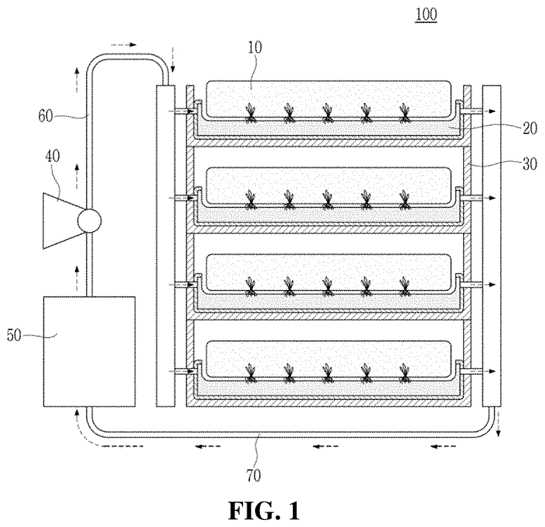

### DETAILED DESCRIPTION OF EXEMPLARY EMBODIMENTS

Hereinafter, the present invention will be described in detail by way of exemplary embodiments. However, the exemplary embodiments are merely illustrative of the present invention and are not intended to limit the present invention thereto.

### Preparation Example 1. Preparation of Sample Such as Green Barley Extract

1-1. Preparation of Green Barley Extract Using Sprout Cultivator (Two Weeks of Cultivation)

In a greenhouse made of glass or vinyl, a plurality of trays having small holes for allowing the roots of sprouts to descend, cultivation plates provided under each of the trays and including waterways for circulating water or a cultivation solution, and a stacking rack (support) enabling the cultivation plates to be stacked were installed. Subsequently, a cultivation solution circulator for circulating water or the cultivation solution through the cultivation plates, a water tank for storing the water or cultivation solution to be supplied to the cultivation plates by the cultivation solution circulator, a first transfer pipe for transferring the water or cultivation solution from the water tank to the cultivation plates, and a second transfer pipe for transferring the water or cultivation solution from the cultivation plates to the water tank were installed, and thereby a sprout cultivator in which water or a cultivation solution is circulated was provided.

Germinated green barley was planted on sponges in the trays so that it was fixed to the sponges, and after the trays were stacked in multiple layers using the stacking rack, the temperature inside the sprout cultivator was maintained at 24 to 27° C., and the green barley sprouts were grown for two weeks until harvested.

The harvested green barley sprouts were washed with purified water, and the whole green barley plant dried at 50° C. was coarsely pulverized and then extracted with a solvent (50 vol % EtOH) 20 times the plant amount for two hours at a constant temperature of 60° C. The resulting liquid extract was centrifuged to collect the extract supernatant, and after the extract was filtered with a 0.2 μm filter, the green barley extract was freeze-dried.

1-2. Preparation of Green Barley Extract Using Sprout Cultivator (Four Weeks of Cultivation)

Green barley extract was obtained in the same manner as in Preparation Example 1 except that sprouts grown for four weeks in the sprout cultivator were used.

1-3. Preparation of Green Barley Extract Using Sprout Cultivator (Six Weeks of Cultivation)

Green Barley Extract was Obtained in the Same Manner as in Preparation Example 1 except that sprouts grown for six weeks in the sprout cultivator were used.

1-4. Preparation of Open-Field Green Barley Extract (Comparative Example)

Green barley extract was obtained in the same manner as in Preparation Example 1 except that sprouts grown for four weeks in an open field in the region of Andeok-myeon, Seogwipo-si, Jeju-do, Korea were used instead of sprouts cultivated in the sprout cultivator.

### Preparation Example 2. Preparation of Saponarin According to Cultivation Period

For use in the experiment, 10 mg of saponarin was purchased from EXTRASYNTHESE. Subsequently, the saponarin was dissolved in autoclaved distilled water, filtered with a 0.2 μM syringe filter, diluted in a cell culture solution, and then used to treat cells.

High-Performance Liquid Chromatography (HPLC) Analysis Conditions for Saponarin
Column: Kromasil C18
Column temperature: 30° C.
Injection volume: 5 μL
Flow rate: 1.0 mL/min
Mobile phase: A=5% acetic acid in water at 80%; D=Methanol at 20%
Gradient: No gradient. A=80% and D=20% from 0 minutes
Detector: UV 270 nm

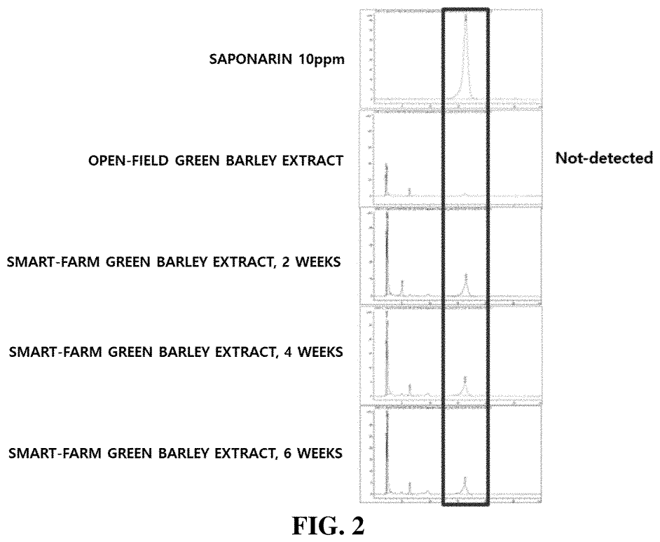

As a result of the analysis, it was found that the green barley extract prepared according to Preparation Example 1 had a high saponarin content (FIG. 2).

### Preparation Example 3. Preparation of Saponarin, Luteolin, and Apigenin

The green barley extract prepared in Preparation Example 1 was prepared at 5,000 ppm and analyzed. A standard mixture was prepared at 1,000 ppm using an MeOH/pyridine mixed solvent, and the standard mixture was diluted to 0.5 to 100 ppm and analyzed.

HPLC Conditions
Instrument: 6410 Triple Quadrupole (LC-MS/MS) commercially available from Agilent
Column: Kromasil C18 (3.0 mm×150 mm, 3.0 μm)
Solvent: A=0.1% formic acid in water; B=0.1% formic acid in acetonitrile
Flow rate: 0.4 mL/min
Injection volume: 5 μL
Gradient:

| — | Time (min) | B % |
|---|---|---|
| 1 | 0 | 5 |
| 2 | 30 | 100 |
| 3 | 32 | 100 |

MS Conditions
Ionization Mode: ESI, scan mode
Gas temperature (° C.): 350
Capillary voltage (V): 4,000
Nebulizer (psig): 40
Fragmentor (V): 135

When a calibration curve was plotted in the component concentration range of 0.5 to 5 μg/mL or 0.5 to 10 μg/mL, the correlation value (R²) was 0.9972 or more, indicating good linearity (Table 1).

TABLE 1

| | | | Correlation |
|---|---|---|---|
| | | Linear range | coefficient |
| Compound | Regression equation | (μg/mL) | (R²) |
| Saponarin | y = 146,054.9216x + | 0.5-10 | 1.0000 |
| | 9,396.9484 | | |
| Luteolin | y = 724,873.5738x + | 0.5-5 | 0.9998 |
| | 286,829.6066 | | |
| Apigenin | y = 972,097.8443x + | 0.5-5 | 0.9972 |
| | 545,760.1639 | | |

As a result of quantitative analysis, it was confirmed that about 33.58 μg of saponarin per 1 mg of the green barley extract was detected.

TABLE 2

| | Amount per 1 mg of sample (μg) | | |
|---|---|---|---|
| Compound | Saponarin | Luteolin | Apigenin |
| Content | 33.58 | N.D. | N.D. |

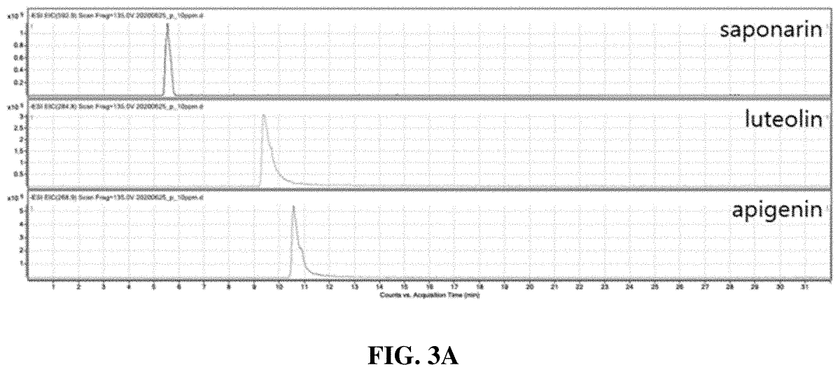

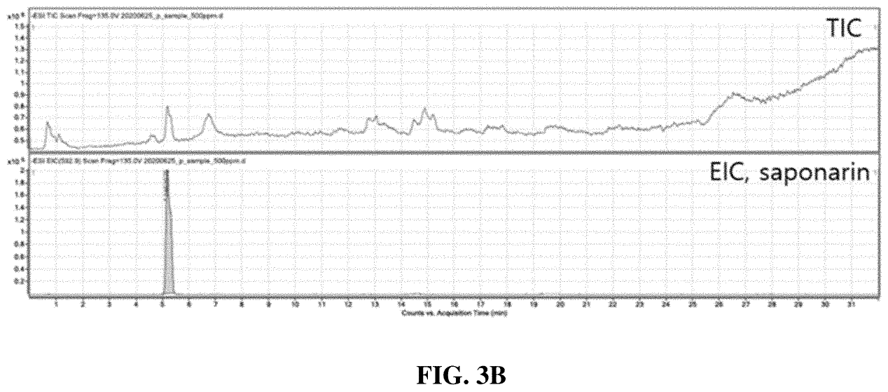

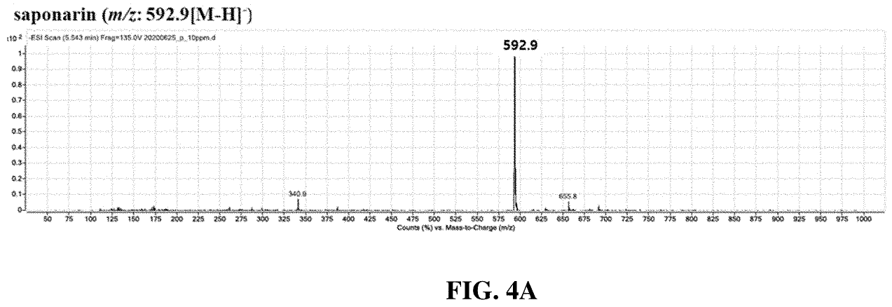

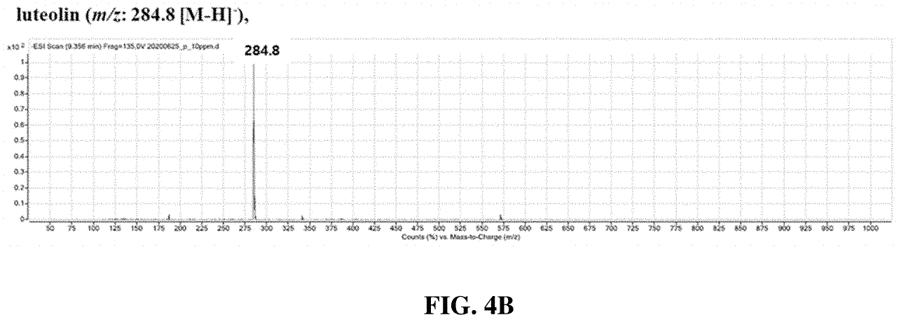

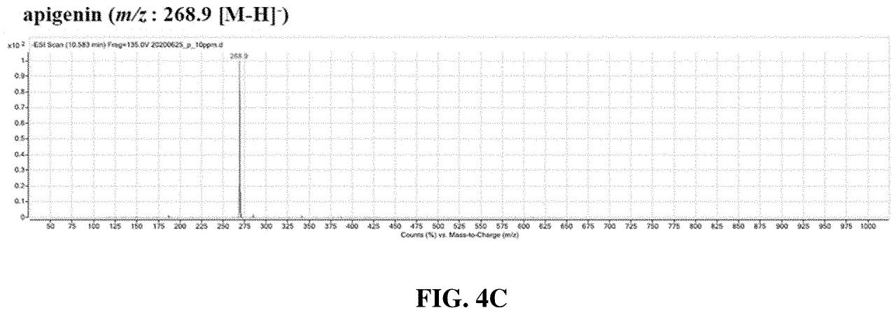

The amounts of saponarin, luteolin, and apigenin in the green barley extract were measured as shown in Table 2, and it was found that the saponarin content was high (FIGS. 3 and 4).

### Example 1. Composition Including Ceramide EOP and Green Barley Extract

In the present invention, ceramide EOP in a dissolved state in ethanol or a polar solution was obtained.

To the dissolved ceramide, the green barley extract prepared through cultivation according to Preparation Example 1 was added at the ratio of 1:0.05 to 1:3, and thereby a composition including ceramide and a green barley extract was obtained.

### Example 2. Composition Including Ceramide NS and Green Barley Extract

In the present invention, ceramide NS in a dissolved state in ethanol or a polar solution was obtained.

The mixing ratio of the ceramide NS and the green barley extract was the same as the mixing ratio in Example 1.

### Example 3. Composition Including Ceramide NP and Green Barley Extract

In the present invention, ceramide NP in a dissolved state in ethanol or a polar solution was obtained.

The mixing ratio of the ceramide NP and the green barley extract was the same as the mixing ratio in Example 1.

### Example 4. Composition Including Ceramide AS and Green Barley Extract

In the present invention, ceramide AS in a dissolved state in ethanol or a polar solution was obtained.

The mixing ratio of the ceramide AS and the green barley extract was the same as the mixing ratio in Example 1.

### Example 5. Composition Including Ceramide AP and Green Barley Extract

In the present invention, ceramide AP in a dissolved state in ethanol or a polar solution was obtained.

The mixing ratio of the ceramide AP and the green barley extract was the same as the mixing ratio in Example 1.

### Example 6. Green Barley Extract

A green barley extract was obtained in the same manner as in Example 1.

### Comparative Example 1. Ceramide EOP

Ceramide EOP in a dissolved state in ethanol or a polar solution was obtained.

### Comparative Example 2. Ceramide NS

Ceramide NS in a dissolved state in ethanol or a polar solution was obtained.

### Comparative Example 3. Ceramide NP

Ceramide NP in a dissolved state in ethanol or a polar solution was obtained.

### Comparative Example 4. Ceramide AS

Ceramide AS in a dissolved state in ethanol or a polar solution was obtained.

### Comparative Example 5. Ceramide AP

Ceramide AP in a dissolved state in ethanol or a polar solution was obtained.

TABLE 3

| | | | | | | Green |
|---|---|---|---|---|---|---|
| | Ceramide | Ceramide | Ceramide | Ceramide | Ceramide | barley |
| | EOP | NS | NP | AS | AP | extract |
| | weight | weight | weight | weight | weight | weight |
| Classification | (%) | (%) | (%) | (%) | (%) | (%) |
| Example | 99 | — | — | — | — | 1 |
| 1 | | | | | | |
| Example | — | 99 | — | — | — | 1 |
| 2 | | | | | | |
| Example | — | — | 99 | — | — | 1 |
| 3 | | | | | | |
| Example | — | — | — | 99 | — | 1 |
| 4 | | | | | | |
| Example | — | — | — | — | 99 | 1 |
| 5 | | | | | | |
| Example | — | — | — | — | — | 100 |
| 6 | | | | | | |
| Comparative | 100 | — | — | — | — | — |
| Example | | | | | | |
| 1 | | | | | | |
| Comparative | — | 100 | — | — | — | — |
| Example | | | | | | |
| 2 | | | | | | |
| Comparative | — | — | 100 | — | — | — |
| Example | | | | | | |
| 3 | | | | | | |
| Comparative | — | — | — | — | — | — |
| Example | | | | | | |
| 4 | | | | | | |
| Comparative | — | — | — | — | — | — |
| Example | | | | | | |
| 5 | | | | | | |

### Experimental Example 1. Culture of Keratinocytes

HaCaT cells, which are keratinocytes, were acquired from Dr. C. G. Hyun (Jeju National University, Korea), cultured in a 37° C., 5% CO₂ thermostat using Dulbecco's Modified Eagle's Medium (DMEM) containing 100 units/ml penicillin-streptomycin and 10% fetal bovine serum (FBS), and subcultured at three- to four-day intervals.

### Experimental Example 2. Cytotoxicity Evaluation (EZ-Cytox Assay)

In order to evaluate the safety of Examples 1 to 6 for the skin, cytotoxicity evaluation was performed using EZ-cytox assay. EZ-cytox assay is a representative method of measuring cell viability using the principle that orange, water-soluble formazan is generated when a substrate reacts with the dehydrogenase of living cells.

Specifically, in order to evaluate the toxicity to keratinocytes, the HaCaT cells were dispensed into a 96-well plate at 5×10⁴ cells/mL using DMEM containing 10% FBS and reacted under the conditions of 37° C. and 5% CO₂ for 18 hours, and absorbance at 450 nm was measured using a microplate reader. The average absorbance for each sample group was determined and compared with the absorbance of the control to evaluate cell viability. The result thereof is shown in Table 4.

TABLE 4
Cell viability (% with respect to control)

Concentration (μg/ml)

| | 0 | 12.5 | 25 | 50 | 100 |
|---|---|---|---|---|---|
| Example 1 | 100 | 114 | 122 | 126 | 122 |
| Example 2 | 100 | 112 | 120 | 124 | 124 |
| Example 3 | 100 | 115 | 122 | 127 | 126 |
| Example 4 | 100 | 114 | 120 | 125 | 120 |
| Example 5 | 100 | 116 | 123 | 124 | 124 |
| Example 6 | 100 | 107.38 | 114.87 | 118.66 | 120.74 |

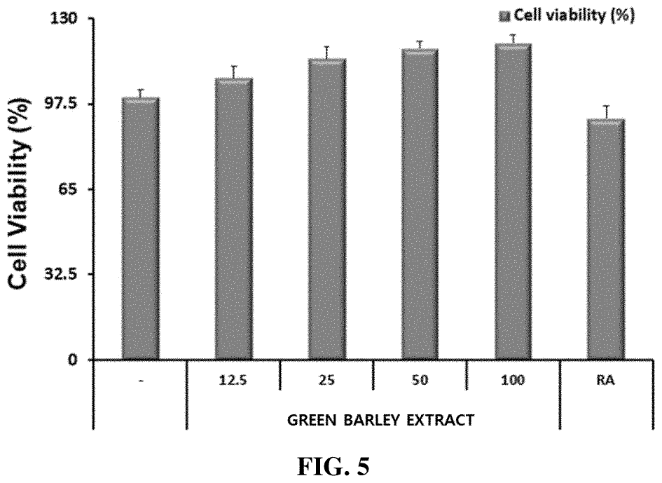

The result confirms that in the case of the ceramide and green barley extract of Examples 1 to 6, excellent cell viability was observed regardless of concentration, and safety was high in view of low toxicity to the keratinocytes (FIG. 5).

### Experimental Example 3. Evaluation of Skin Barrier Strengthening and Skin Moisturizing Effects of Saponarin

In the present invention, an experiment was conducted to evaluate the effect of saponarin isolated in Preparation Examples 2 and 3 on the expression levels of moisturizing factors.

3-1. Evaluation of Skin Barrier Strengthening Effect of Saponarin

HaCaT cells were dispensed into a 24-well plate at 1.0×10⁵ cells/mL and cultured under the conditions of 37° C. and 5% CO₂ for 18 hours. After the medium was replaced with serum-free DMEM, the cells were treated with a sample and then cultured for 24 hours. Subsequently, the culture solution was removed, and each group was washed with PBS and then treated with PBS containing no drugs that could interfere with the quantification of proteins. The cells were lysed through repeated switching between low-temperature incubation and room-temperature incubation, and the proteins were collected. The quantification was performed, using a Filaggrin-ELISA kit (Elabscience Biotechnology Co., Ltd.), according to the method specified by the manufacturer.

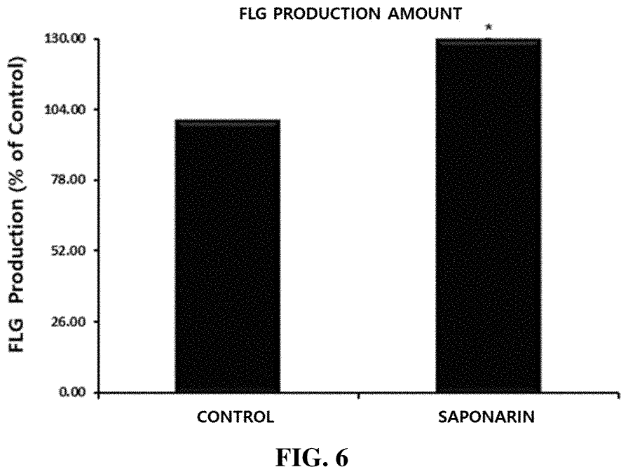

The result confirms that in view of the fact that saponarin increased the expression level of FLG by 29.87% compared to the control, saponarin is a material highly effective for protecting the skin barrier (FIG. 6).

3-2. Evaluation of Skin Moisturizing Effect of Saponarin

HaCaT cells were dispensed into a 24-well plate at 1.0×10⁵ cells/mL and cultured under the conditions of 37° C. and 5% CO₂ for 18 hours. After the medium was replaced with serum-free DMEM, the cells were treated with a sample and then cultured for 24 hours. Subsequently, the culture medium was collected and centrifuged at 15,000 rpm for five minutes, and the supernatant was collected and stored frozen (−20° C.) until quantified. Enzyme-linked immunosorbent assay (ELISA) was conducted, using a hyaluronic acid ELISA kit (Elabscience Biotechnology Co., Ltd.), according to the method specified by the manufacturer.

In addition, HaCaT cells were dispensed into a 24-well plate at 1.0×10⁵ cells/mL and then cultured under the conditions of 37° C. and 5% CO₂ for 18 hours. After the medium was replaced with serum-free DMEM, the cells were treated with a sample and then cultured for 24 hours. Subsequently, the culture solution was removed, and each group was washed with PBS and then treated with PBS containing no drugs that could interfere with the quantification of proteins. The cells were lysed through repeated switching between low-temperature incubation and room-temperature incubation, and the proteins were collected. The quantification was performed, using an AQP3-ELISA kit (Elabscience Biotechnology Co., Ltd.), according to the method specified by the manufacturer.

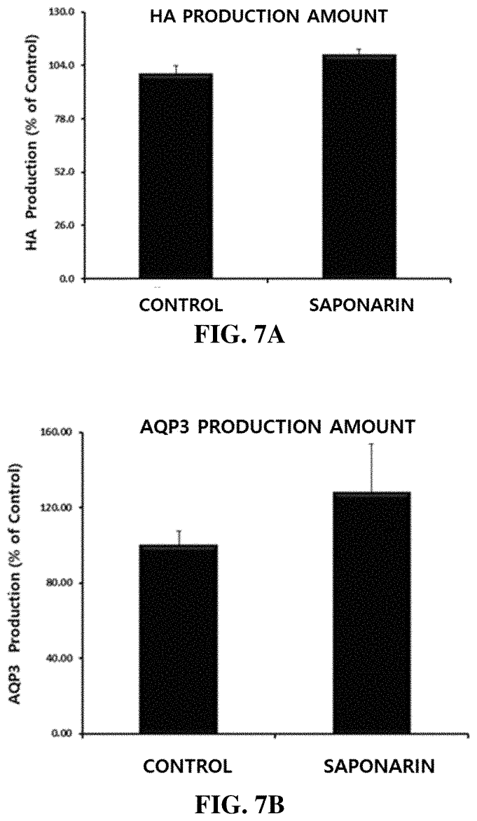

The result confirms that saponarin significantly increased the production of skin moisturizing factors, and that saponarin is a material that is effective for moisturizing the skin by promoting the production of HA and AQP3 (FIGS. 7A-7B).

### Experimental Example 4. Evaluation of Skin Barrier Strengthening Effect of Green Barley Extract and Ceramide

In order to evaluate the skin barrier strengthening effect of Examples 1 to 6 and Comparative Examples 1 to 5, an experiment was conducted to evaluate the effect of increasing the production of FLG.

The production amount of FLG was measured in the same manner as in Experimental Example 3-1.

TABLE 5
Increase in FLG production (%)

| — | Concentration | % |
|---|---|---|
| Control | — | 100 |
| Example 1 | 50 | 130.5 |
| Example 2 | 50 | 130.2 |
| Example 3 | 50 | 145.7 |
| Example 4 | 50 | 134.0 |
| Example 5 | 50 | 137.5 |
| Example 6 | 50 | 129.92 |
| Comparative | 50 | 119.5 |
| Example 1 | | |
| Comparative | 50 | 116.7 |
| Example 2 | | |
| Comparative | 50 | 115.5 |
| Example 3 | | |
| Comparative | 50 | 122.4 |
| Example 4 | | |
| Comparative | 50 | 129.9 |
| Example 5 | | |

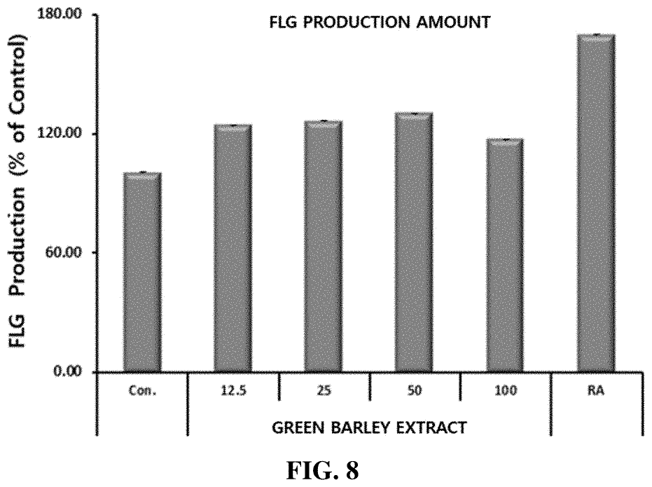

As shown in Table 5, since Examples 1 to 6 significantly increased the production of the FLG factor compared to Comparative Examples 1 to 5, it is confirmed that the ceramide and green barley extract of the present invention are suitable for use in skin barrier strengthening (FIG. 8). In addition, in view of the fact that the FLG production amount was particularly high with the use of the composition including ceramide NP and the green barley extract, it can be seen that the composition including ceramide NP and the green barley extract is highly effective for strengthening the skin barrier.

### Experimental Example 5. Evaluation of Moisturizing Effect of Composition Including Green Barley Extract and Ceramide

5-1. Evaluation of HA (Moisturizing Factor) Production Increasing Effect

In order to evaluate the moisturizing effect of Examples 1 to 6 and Comparative Examples 1 to 5, an experiment was conducted to evaluate an HA production increasing effect.

The HA production amount was measured in the same manner as in Experimental Example 3-2.

TABLE 6
Increase in HA production (%)

| — | Concentration | % |
|---|---|---|
| Control | — | 100 |
| Example 1 | 50 | 124.55 |
| Example 2 | 50 | 124.60 |
| Example 3 | 50 | 129.95 |
| Example 4 | 50 | 126.37 |
| Example 5 | 50 | 127.00 |
| Example 6 | 50 | 121.58 |
| Comparative | 50 | 93.0 |
| Example 1 | | |
| Comparative | 50 | 94.5 |
| Example 2 | | |
| Comparative | 50 | 93.2 |
| Example 3 | | |
| Comparative | 50 | 94.6 |
| Example 4 | | |
| Comparative | 50 | 95.2 |
| Example 5 | | |

5-2. Evaluation of AQP3 (Moisturizing Factor) Production Increasing Effect

In order to evaluate the moisturizing effect of Examples 1 to 6 and Comparative Examples 1 to 5, an experiment was conducted to evaluate an AQP3 production increasing effect.

The AQP3 production amount was measured in the same manner as in Experimental Example 3-2.

TABLE 7
Increase in AQP3 production (%)

| — | Concentration | % |
|---|---|---|
| Control | — | 100 |
| Example 1 | 50 | 124.68 |
| Example 2 | 50 | 124.40 |
| Example 3 | 50 | 134.50 |
| Example 4 | 50 | 127.00 |
| Example 5 | 50 | 131.74 |
| Example 6 | 50 | 128.00 |
| Comparative | 50 | 117.5 |
| Example 1 | | |
| Comparative | 50 | 118.5 |
| Example 2 | | |
| Comparative | 50 | 114.6 |
| Example 3 | | |
| Comparative | 50 | 114.5 |
| Example 4 | | |
| Comparative | 50 | 113.7 |
| Example 5 | | |

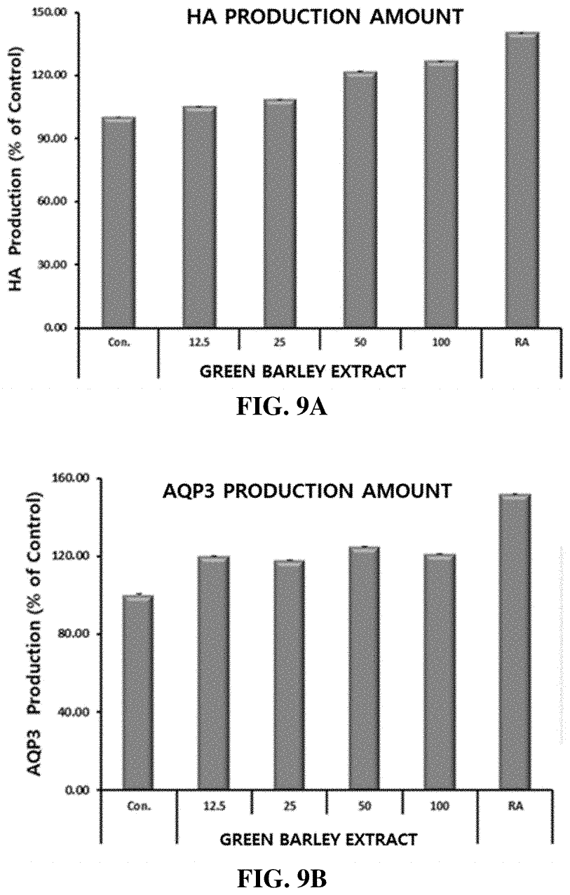

As shown in Tables 6 and 7, it was confirmed that the green barley extract promotes the production of HA and AQP3 factors FIGS. 9A-9B), and further, ceramide works synergistically with the green barley extract to promote the production of skin moisturizing factors.

### Experimental Example 6. Human Application Test Results According to Types of Formulation for Evaluating Moisturizing and Skin Barrier Strengthening Effects of Composition Including Green Barley Extract and Ceramide

In order to evaluate soothing gel, lotion, and cream formulations including a composition including the ceramide and green barley extract of the present invention in the areas of moisture retention ability, superficial moisturization, deep skin moisturization, transepidermal moisture loss, and a skin moisture barrier improving effect, the inventors requested the OATC Skin Clinical Trials Center to perform a human application test, and the test was conducted in accordance with the cosmetics guidelines of Korea's Ministry of Food and Drug Safety, Declaration of Helsinki, and OATC Skin Clinical Trials Center's standard operating procedure (SOP).

The test was conducted with 32 test subjects without any dropouts. The test was conducted for a total of four weeks, and the data regarding the following seven test items was collected at time points such as before use, immediately after use, after 48 hours of use, and after four weeks of use: 1) 48-hour moisture retention ability, 2) superficial moisturization, 3) deep skin moisturization, 4) transepidermal moisture loss, 5) skin moisture barrier, 6) subjective questionnaire evaluation conducted by test subjects, and 7) adverse reactions sensed by test subjects and testers.

Ingredients of the soothing gel, lotion, and cream formulations used for the human application test are listed in Tables 8 to 10, respectively.

TABLE 8
Soothing gel

| Ingredient name | % (W/W) |
|---|---|
| Glycerin | 8.0 |
| Panthenol | 5.0 |
| Caprylic/capric triglyceride | 5.0 |
| Cetearyl alcohol | 3.0 |
| 1,2-Hexanediol | 1.0 |
| Ammonium acryloyldimethyltaurate/VP copolymer | 0.4 |
| Betaine | 0.3 |
| Ceramide NP | 0.1 |
| Hydroxyacetophenone | 0.3 |
| Carbomer | 0.3 |
| Arginine | 0.3 |
| Butylene glycol | 0.3 |
| Allantoin | 0.1 |
| Aloe vera leaf juice | 0.1 |
| Disodium EDTA | 0.03 |
| Green barley extract | 0.01 |
| Madecassoside | 0.01 |
| Centella leaf extract | 0.005 |
| Lotus seed extract | 0.0022 |
| Lens esculenta extract | 0.0022 |
| Quinoa seed extract | 0.0022 |
| Carob fruit extract | 0.0022 |
| Asiaticoside | 0.0020 |
| Centaurea cyanus flower extract | 0.0010 |
| Clary extract | 0.0010 |
| Lavender flower extract | 0.0010 |
| Hyacinth whole plant extract | 0.0010 |
| Matricaria flower extract | 0.0010 |
| Borage extract | 0.0010 |
| Ethylhexylglycerin | 0.0100 |
| Ethyl hexanediol | 0.001 |
| Purified water | To 100 |

TABLE 9
Lotion

| Ingredient name | Content (g) |
|---|---|
| Glycerin | 10.748 |
| Caprylic/capric triglyceride | 5.0 |
| Panthenol | 5.0 |
| Sunflower seed oil | 3.9 |
| Caprylyl methicone | 1.5 |
| Cetearyl alcohol | 1.14 |
| 1,2-Hexanediol | 1.0294 |
| Cetearyl olivate | 0.78 |
| Sorbitan olivate | 0.52 |
| Sorbitan stearate | 0.40 |
| Stearic acid | 0.40 |
| Hydroxyacetophenone | 0.40 |
| Hydrogenated olive oil | 0.325 |
| Cacao seed butter | 0.30 |
| Cetearyl glucoside | 0.260 |
| Carbomer | 0.250 |
| Olive oil | 0.1375 |
| Butylene glycol | 0.1191 |
| Ceramide NP | 0.1 |
| Avocado oil | 0.1 |
| Tromethamine | 0.1 |
| Sodium phytate | 0.05 |
| Allantoin | 0.05 |
| Ethylhexylglycerin | 0.05 |
| Olive oil unsaponifiable | 0.0375 |
| Glyceryl acrylate/acrylic acid copolymer | 0.030 |
| Lens esculenta extract | 0.022 |
| Lotus seed extract | 0.022 |
| Carob fruit extract | 0.022 |
| Quinoa seed extract | 0.022 |
| Matricaria flower extract | 0.01005 |
| Lavender flower extract | 0.01 |
| Borage extract | 0.01 |
| Green barley extract | 0.01 |
| Centaurea cyanus flower extract | 0.01 |
| Clary extract | 0.01 |
| Hyacinth whole plant extract | 0.01 |
| Centella extract | 0.00052 |
| Pinus sylvestris leaf extract | 0.00010 |
| Wild bean sprout extract | 0.00010 |
| Spanish licorice root extract | 0.00010 |
| Beta-glucan | 0.000040 |
| Purified water | To 100 |

TABLE 10
Cream

| Ingredient name | % (W/W) |
|---|---|
| Caprylic/capric triglyceride | 10 |
| Glycerin | 9 |
| Panthenol | 5 |
| Hydrogenated polydecene | 5 |
| Pentylene glycol | 3 |
| Cetyl alcohol | 2 |
| Glycosyl trehalose | 2 |
| Polyglyceryl-3-distearate | 1 |
| 1,2-Hexanediol | 1 |
| Vinyl dimethicone | 1 |
| Propanediol | 1 |
| Bacillus/soybean ferment extract | 1 |
| Hydrogenated starch hydrolysate | 1 |
| Ceramide NP | 0.5 |
| Cetearyl olivate | 0.5 |
| Candida bombicola/glucose/methyl rapeseedate ferment | 0.3 |
| Sunflower seed oil | 0.3 |
| Glyceryl stearate | 0.3 |
| Sorbitan olivate | 0.3 |
| Carbomer | 0.2 |
| Tromethamine | 0.2 |
| Butylene glycol | 0.2 |
| Acrylate/C10-C30 alkyl acrylate crosspolymer | 0.2 |
| Glyceryl stearate citrate | 0.1 |
| Sodium hyaluronate | 0.1 |
| Ethylhexylglycerin | 0.05 |
| Disodium EDTA | 0.02 |
| Green barley extract | 0.01 |
| Lens esculenta extract | 0.0022 |
| Carob fruit extract | 0.0022 |
| Lotus seed extract | 0.0022 |
| Quinoa seed extract | 0.0022 |
| Lavender flower extract | 0.0010 |
| Clary extract | 0.0010 |
| Hyacinth whole plant extract | 0.0010 |
| Matricaria flower extract | 0.0010 |
| Borage extract | 0.0010 |
| Centaurea cyanus flower extract | 0.0010 |
| Purified water | To 100 |

6-1. Evaluation of Moisturizing Effect of Soothing Gel/Lotion/Cream Formulations

A total of 32 test subjects were asked to apply the soothing gel, lotion, and cream formulations described in Tables 8 to 10 to the face and the whole body for four weeks. The 48-hour moisture retention ability was measured, using Epsilon E100, in the same left forearm area before, immediately after, and after 48 hours of use of a product in the above-described formulations, and the measured value was the average dielectric constant of the entire skin represented by ε. Here, larger measurement values indicate a stronger 48-hour moisture retention ability.

The superficial moisturization was evaluated, using Epsilon E100, in the same right cheek area before and after four weeks of use of a product in the above-described formulations, and the measured value was the average dielectric constant of the entire skin represented by ε. Here, larger measurement values indicate a greater improvement in superficial moisturization.

The deep skin moisturization was evaluated, using MoistureMeterD, in the same right cheek area before and after four weeks of use of a product in the above-described formulations, and the measured value was a tissue dielectric constant (TDC), which is a value proportional to the total amount of moisture in the tissue. Here, larger measurement values indicate a greater improvement in deep skin moisturization.

The amount of transepidermal moisture loss was measured, using Tewameter® TM300, in the same right cheek area before and after four weeks of use of a product in the above-described formulations. The measured value was a g/m²/h value representing the degree of moisture release from the skin epidermis, and larger measurement values indicate a greater improvement in transepidermal moisture loss. The result of human application test is shown in the following Tables 11 to 13.

Soothing gel

| | | | p-value | | |
|---|---|---|---|---|---|
| | | | Within- | | |
| | | Degree of | subject | | |
| | Measurement | improvement | effect | Post hoc | |
| Test item | time | (%) | analysis | analysis | Result |
| 48-hour | Immediately | 169.62 | 0.000*** | 0.000*** | Moisture |
| moisture | after use | | | | retention |
| retention | After 48 | 65.88 | | 0.000*** | ability is |
| ability | hours of | | | | exhibited |
| | use | | | | after 48 |
| | | | | | hours of use |

| | | | | |
|---|---|---|---|---|
| Superficial | After four | 32.10 | 0.000*** | Improvement |
| moisturization | weeks of | | | in superficial |
| | use | | | moisturization |
| | | | | is observed |
| | | | | after four |
| | | | | weeks of use |
| Deep skin | After four | 13.01 | 0.000*** | Improvement |
| moisturization | weeks of | | | in deep skin |
| | use | | | moisturization |
| | | | | is observed |
| | | | | after four |
| | | | | weeks of use |
| Transepidermal | After four | 15.89 | 0.000*** | Improvement |
| moisture | weeks of | | | in trans- |
| loss | use | | | epidermal |
| | | | | moisture |
| | | | | loss is |
| | | | | observed |
| | | | | after four |
| | | | | weeks of use |

| | |
|---|---|
| Conclusion | It is determined that the product helps the skin to retain moisture for |
| | 48 hours with a single use and helps to improve superficial |
| | moisturization, deep skin moisturization, and transepidermal |
| | moisture loss with four weeks of use. |

TABLE 12
Lotion

| | | | p-value | | |
|---|---|---|---|---|---|
| | | | Within- | | |
| | | Degree of | subject | | |
| | Measurement | improvement | effect | Post hoc | |
| Test item | time | (%) | analysis | analysis | Result |
| 48-hour | Immediately | 261.85 | 0.000*** | 0.000*** | Moisture |
| moisture | after use | | | | retention |
| retention | After 48 | 84.86 | | 0.000*** | ability is |
| ability | hours of | | | | exhibited |
| | use | | | | after 48 |
| | | | | | hours of use |

| | | | | |
|---|---|---|---|---|
| Superficial | After four | 20.53 | 0.000*** | Improvement |
| moisturization | weeks of | | | in superficial |
| | use | | | moisturization |
| | | | | is observed |
| | | | | after four |
| | | | | weeks of use |
| Deep skin | After four | 7.24 | 0.000*** | Improvement |
| moisturization | weeks of | | | in deep skin |
| | use | | | moisturization |
| | | | | is observed |
| | | | | after four |
| | | | | weeks of use |
| Transepidermal | After four | 9.24 | 0.031*** | Improvement |
| moisture | weeks of | | | in trans- |
| loss | use | | | epidermal |
| | | | | moisture |
| | | | | loss is |
| | | | | observed |
| | | | | after four |
| | | | | weeks of use |

| | |
|---|---|
| Conclusion | It is determined that the product helps the skin to retain moisture for |
| | 48 hours with a single use and helps to improve superficial |
| | moisturization, deep skin moisturization, and transepidermal moisture |
| | loss with four weeks of use. |

TABLE 13
Cream

| | | | p-value | | |
|---|---|---|---|---|---|
| | | | Within- | | |
| | | Degree of | subject | | |
| | Measurement | improvement | effect | Post hoc | |
| Test item | time | (%) | analysis | analysis | Result |
| 48-hour | Immediately | 242.05 | 0.000*** | 0.000*** | Moisture |
| moisture | after use | | | | retention |
| retention | After 48 | 71.77 | | 0.000*** | ability is |
| ability | hours of | | | | exhibited |
| | use | | | | after 48 |
| | | | | | hours of use |

| | | | | |
|---|---|---|---|---|
| Superficial | After four | 31.85 | 0.000*** | Improvement |
| moisturization | weeks of | | | in superficial |
| | use | | | moisturization |
| | | | | is observed |
| | | | | after four |
| | | | | weeks of use |
| Deep skin | After four | 12.68 | 0.000*** | Improvement |
| moisturization | weeks of | | | in deep skin |
| | use | | | moisturization |
| | | | | is observed |
| | | | | after four |
| | | | | weeks of use |
| Transepidermal | After four | 10.57 | 0.000*** | Improvement |
| moisture | weeks of | | | in trans- |
| loss | use | | | epidermal |
| | | | | moisture |
| | | | | loss is |
| | | | | observed |
| | | | | after four |
| | | | | weeks of use |

| | |
|---|---|
| Conclusion | It is determined that the product helps the skin to retain moisture for |
| | 48 hours with a single use and helps to improve superficial |
| | moisturization, deep skin moisturization, and transepidermal moisture |
| | loss with four weeks of use. |

According to the results shown in Tables 11 to 13, in the case of all of the soothing gel, lotion, and cream formulations, all of 48-hour moisture retention ability, superficial moisturization, and deep skin moisturization were significantly improved, and the amount of transdermal moisture loss was significantly decreased, to 15.89%, after four weeks of use. These results suggest that cosmetic compositions in the above-described formulations have remarkable effects on deep skin moisturization, superficial moisturization, and moisture retention ability.

6-2. Evaluation of Skin Barrier Improving Effect of Soothing Gel/Lotion/Cream Formulations

TABLE 14
Soothing gel

| | | | p-value | | |
|---|---|---|---|---|---|
| | | | Within- | | |
| | | Degree of | subject | | |
| | Measurement | improvement | effect | Post hoc | |
| Test item | time | (%) | analysis | analysis | Result |

| | | | | | |
|---|---|---|---|---|---|
| Skin | Superficial | After four | 32.10 | 0.000*** | Improvement |
| moisture | moisturization | weeks of | | | in skin |
| barrier | | use | | | moisture |
| | Transepidermal | After four | 15.89 | 0.000*** | barrier is |
| | moisture | weeks of | | | observed |
| | loss | use | | | after four |
| | | | | | weeks of use |

| | |
|---|---|
| Conclusion | It is determined that the product helps to improve skin moisture barrier |
| | with four weeks of use. |

TABLE 15
Lotion

| | | | p-value | | |
|---|---|---|---|---|---|
| | | | Within- | | |
| | | Degree of | subject | | |
| | Measurement | improvement | effect | Post hoc | |
| Test item | time | (%) | analysis | analysis | Result |

| | | | | | |
|---|---|---|---|---|---|
| Skin | Superficial | After four | 20.53 | 0.000*** | Improvement |
| moisture | moisturization | weeks of | | | in skin |
| barrier | | use | | | moisture |
| | Transepidermal | After four | 9.24 | 0.031*** | barrier is |
| | moisture | weeks of | | | observed |
| | loss | use | | | after four |
| | | | | | weeks of use |

| | |
|---|---|
| Conclusion | It is determined that the product helps to improve skin moisture barrier |
| | with four weeks of use. |

TABLE 16
Cream

| | | | p-value | | |
|---|---|---|---|---|---|
| | | | Within- | | |
| | | Degree of | subject | | |
| | Measurement | improvement | effect | Post hoc | |
| Test item | time | (%) | analysis | analysis | Result |

| | | | | | |
|---|---|---|---|---|---|
| Skin | Superficial | After four | 31.85 | 0.000*** | Improvement |
| moisture | moisturization | weeks of | | | in skin |
| barrier | | use | | | moisture |
| | Transepidermal | After four | 10.57 | 0.000*** | barrier is |
| | moisture | weeks of | | | observed |
| | loss | use | | | after four |
| | | | | | weeks of use |

| | |
|---|---|
| Conclusion | It is determined that the product helps to improve skin moisture barrier |
| | with four weeks of use. |

According to the results shown in Tables 14 to 16, in the case of all of the soothing gel, lotion, and cream formulations, the skin moisture barrier protecting effect was significantly increased, and the skin barrier improving effect of the soothing gel formulation was particularly excellent. These results suggest that the cosmetic composition of the present invention is highly effective for maintaining skin moisturization by strengthening the skin barrier.

### Experimental Example 7. Human Application Test Results According to Types of Formulation for Evaluating Suitability of Use of Green Barley Extract and Ceramide on Sensitive Skin

A test was conducted to evaluate the suitability and safety of use of soothing gel, lotion, cream, body wash, and foam cleanser formulations including a composition including the ceramide and green barley extract of the present invention. The inventors requested the OATC Skin Clinical Trials Center to perform a human application test, and the test was conducted in accordance with the cosmetics guidelines of Korea's Ministry of Food and Drug Safety, Declaration of Helsinki, and OATC Skin Clinical Trials Center's SOP.

The soothing gel, lotion, and cream formulations are the same as those described in Tables 8 to 10, and body wash and foam cleanser formulations are described in Tables 17 and 18, respectively.

TABLE 17
Body wash

| Ingredient name | Content (%) |
|---|---|
| Lauryl glucoside | 7.500 |
| Disodium cocoamphodiacetate | 5.505 |
| Sodium chloride | 3.750 |
| Glycerin | 3.0026 |
| Lauryl betaine | 3.00 |
| Citric acid | 0.90 |
| Butylene glycol | 0.2797 |
| Caprylyl glycol | 0.22403 |
| Hydroxyacetophenone | 0.160 |
| Pentylene glycol | 0.160 |
| Tocopherol | 0.050 |
| Disodium EDTA | 0.050 |
| Hexylene glycol | 0.045 |
| Ethylhexylglycerin | 0.03001 |
| Panthenol | 0.010 |
| Allantoin | 0.010 |
| Green tea extract | 0.0049 |
| Spanish licorice root extract | 0.0049 |
| Orange peel oil | 0.0035 |
| Bergamot oil | 0.0035 |
| Philippine orange peel oil | 0.0030 |
| Caprylic/capric triglyceride | 0.0014 |
| 1,2-Hexanediol | 0.00507 |
| Centella leaf extract | 0.0006 |
| Squalane | 0.0006 |
| Olive oil | 0.0005 |
| Hydrogenated lecithin | 0.0005 |
| Shea butter | 0.0004 |
| Ceramide NP | 0.0001 |
| Beta-glucan | 0.00005 |
| Lens esculenta extract | 0.0022 |
| Carob fruit extract | 0.0022 |
| Lotus seed extract | 0.0022 |
| Quinoa seed extract | 0.0022 |
| Lavender flower extract | 0.001 |
| Clary extract | 0.001 |
| Hyacinth whole plant extract | 0.0010 |
| Matricaria flower extract | 0.0010 |
| Borage extract | 0.0010 |
| Centaurea cyanus flower extract | 0.0010 |
| Green barley extract | 0.002 |
| Purified water | To 100 |

TABLE 18
Foam cleanser

| Ingredient name | Content (%) |
|---|---|
| Glycerin | 10.5026 |
| Lauryl hydroxysultaine | 3.8 |
| Disodium cocoyl glutamate | 1.50 |
| Coco-betaine | 2.10 |
| Lauryl betaine | 1.20 |
| Sodium chloride | 1.140 |
| Sodium cocoamphoacetate | 1.00 |
| Panthenol | 1.00 |
| Allantoin | 0.010 |
| Green tea extract | 0.0049 |
| Spanish licorice root extract | 0.0049 |
| Orange peel oil | 0.0035 |
| Bergamot oil | 0.0035 |
| Philippine orange peel oil | 0.0030 |
| Caprylic/capric triglyceride | 0.0014 |
| Centella leaf extract | 0.0006 |
| Squalane | 0.0006 |
| Olive oil | 0.0005 |
| Hydrogenated lecithin | 0.0005 |
| Shea butter | 0.0004 |
| Ceramide NP | 0.0001 |
| Beta-glucan | 0.00005 |
| Lens esculenta extract | 0.0022 |
| Carob fruit extract | 0.0022 |
| Lotus seed extract | 0.0022 |
| Quinoa seed extract | 0.0022 |
| Lavender flower extract | 0.001 |
| Clary extract | 0.001 |
| Hyacinth whole plant extract | 0.001 |
| Matricaria flower extract | 0.001 |
| Borage extract | 0.001 |
| Centaurea cyanus flower extract | 0.001 |
| Green barley extract | 0.002 |
| Lauryl glycoside | 0.75 |
| Citric acid | 0.30 |
| Butylene glycol | 0.2797 |
| Sodium cocoyl glutamate | 0.2500 |
| Sodium lauryl glucose carboxylate | 0.250 |
| Caprylyl glycol | 0.224 |
| Hydroxyacetophenone | 0.160 |
| Pentylene glycol | 0.160 |
| Disodium EDTA | 0.10 |
| Ethylhexylglycerin | 0.05 |
| 1,2-Hexanediol | 0.00507 |
| Purified water | To 100 |

7-1. Evaluation of Safety of Soothing Gel/Lotion/Cream Formulations for Sensitive Skin

In order to classify the test subjects according to skin type, the test subjects were screened by a stinging test to identify those with sensitive skin, wherein steam was applied to their faces for 10 minutes using a steamer to make their skin sensitive, and subsequently, each of 10% lactic acid and distilled water (D.W.) was applied to the nasolabial folds on either side of the nose. The test subjects were observed at two minutes, four minutes, six minutes, eight minutes, and ten minutes after application to determine whether irritation had occurred, and those with irritation only in the area where lactic acid had been applied were classified as test subjects with sensitive skin.

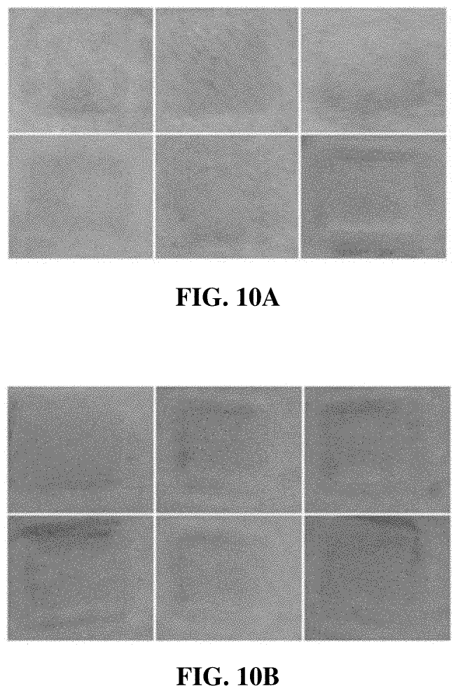

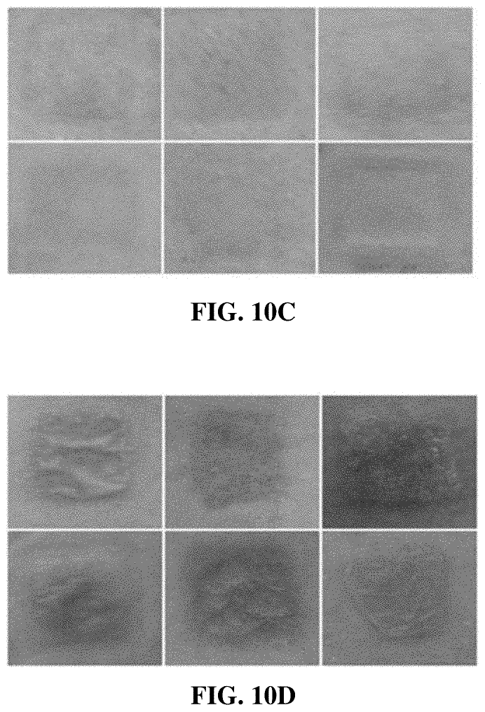

After the test subject's test site was wiped off with 70% ethanol and dried, 25 μl of a test product was applied dropwise to IQ Ultra, which was then attached and fixed to the test site. At 30 minutes, 24 hours, and 48 hours after removing the patch, the level of irritation was evaluated by two experts using, as a standard, the evaluation method devised by Frosch & Kligman and employing the reading standards of the International Contact Dermatitis Research Group (ICDRG). The evaluation criteria are shown in the following Table 19, and the skin irritation levels are shown in FIGS. 10A-10D.

TABLE 19

| | | Types of skin symptoms or |
|---|---|---|
| Irritation level | Score | signs |
| − | 0 | Negative |
| ± | 0.5 | Suspicious skin symptoms |
| | | such as mild erythema |
| + | 1 | Slight erythema with spots |
| | | or spread |
| ++ | 2 | Moderately uniform |
| | | erythema |
| +++ | 3 | Edema and severe |
| | | erythema |
| ++++ | 4 | Intense erythema with |
| | | edema and blisters |
| − (negative) | — | Negative reaction |
| IR (irritant | — | Various other types of |
| reaction) | | irritation reactions |
| | | (including contact |
| | | irritation) |
| NT (not tested) | — | Test is interrupted due to |
| | | the occurrence of irritation |
| | | reactions or other reasons |

In addition, safety zones were established, and an experiment was conducted while using the safety zones as criteria for evaluating the likelihood of causing irritation. The safety zones for the human primary irritation test are shown in the following Table 20.

TABLE 20

| | |
|---|---|
| Product category | Safety zone |

Average reaction index R with z ≤1.00

| | | |
|---|---|---|
| Leave-on type | Toners | 0.87 |
| | Lotions and creams | 1.12 |
| | Foundations | 0.97 |
| | Sunscreens | 0.83 |
| | Lip products | 0.98 |
| | Body lotions and body creams | 0.94 |
| | Face powders and eye | 1.02 |
| | shadows | |
| | Eyeliners and mascaras | 2.96 |
| Tissue-off type | Makeup removers | 1.36 |
| | Packs and massage creams | 1.33 |
| Wash-off type | Facial cleansers | 3.20 |
| | Soaps and bath soaps | 3.98 |
| | Shampoos, conditioners, and | 2.82 |
| | hair treatments | |

In addition, through a single patch test reading, it was evaluated whether a test product caused a reaction in more than 20% of all test subjects or irritation reactions with an irritation level of at least ++ were observed in at least 10% of all test subjects at each reading. Products having these characteristics are expected to be significantly more likely to cause irritation.

In this regard, when a product did not satisfy the safety zone criterion, caused irritation in more than 20% of all test subjects, or irritation reactions with an irritation level of at least ++ were observed in at least 10% of all test subjects at each reading, or two or more of such characteristics were exhibited, the test product was evaluated as a product likely to cause irritation and was evaluated as “non-conforming.”

TABLE 21
Soothing gel

| | Sum of skin | | | | | | | Irritation of | |
|---|---|---|---|---|---|---|---|---|---|
| | irritation | | | | | | Irritation | level ++ or | |
| | reaction | | | Skin | Skin | | in >20% | more in >10% | |
| Sample | intensity | | | irritation | irritation | Safety | of test | of test | |
| name | 1^{st} | 2^{nd} | 3^{rd} | index | rating | zone | subjects | subjects | Conformity |
| Soothing | 0.00 | 0.00 | 0.00 | 0.00 | Not | Conforming | Conforming | Conforming | Conforming |
| gel of | | | | | irritating | | | | |
| present | | | | | | | | | |
| invention | | | | | | | | | |
| D.W. | 0.00 | 0.00 | 0.00 | 0.00 | Not | Conforming | Conforming | Conforming | Conforming |
| (Negative | | | | | irritating | | | | |
| control) | | | | | | | | | |
| 1% SLS | 4.00 | 4.00 | 3.06 | 3.06 | Moderately | Conforming | Non- | Conforming | Non- |
| (Positive | | | | | irritating | | conforming | | conforming |
| control) | | | | | | | | | |

TABLE 22
Lotion

| | Sum of skin | | | | | | | Irritation of | |
|---|---|---|---|---|---|---|---|---|---|
| | irritation | | | | | | Irritation | level ++ or | |
| | reaction | | | Skin | Skin | | in >20% | more in >10% | |
| Sample | intensity | | | irritation | irritation | Safety | of test | of test | |
| name | 1^{st} | 2^{nd} | 3^{rd} | index | rating | zone | subjects | subjects | Conformity |
| Lotion of | 0.00 | 0.00 | 0.00 | 0.00 | Not | Conforming | Confirming | Conforming | Conforming |
| present | | | | | irritating | | | | |
| invention | | | | | | | | | |
| D.W. | 0.00 | 0.00 | 0.00 | 0.00 | Not | Conforming | Conforming | Conforming | Conforming |
| (Negative | | | | | irritating | | | | |
| control) | | | | | | | | | |
| 1% SLS | 4.00 | 4.00 | 3.06 | 3.06 | Moderately | Conforming | Non- | Conforming | Non- |
| (Positive | | | | | irritating | | conforming | | conforming |
| control) | | | | | | | | | |

TABLE 23
Cream

| | Sum of skin | | | | | | | Irritation of | |
|---|---|---|---|---|---|---|---|---|---|
| | irritation | | | | | | Irritation | level ++ or | |
| | reaction | | | Skin | Skin | | in >20% | more in >10% | |
| Sample | intensity | | | irritation | irritation | Safety | of test | of test | |
| name | 1^{st} | 2^{nd} | 3^{rd} | index | rating | zone | subjects | subjects | Conformity |
| Cream of | 0.00 | 0.00 | 0.00 | 0.00 | Not | Conforming | Conforming | Conforming | Conforming |
| present | | | | | irritating | | | | |
| invention | | | | | | | | | |
| D.W. | 0.00 | 0.00 | 0.00 | 0.00 | Not | Conforming | Conforming | Conforming | Conforming |
| (Negative | | | | | irritating | | | | |
| control) | | | | | | | | | |
| 1% SLS | 4.00 | 4.00 | 3.06 | 3.06 | Moderately | Conforming | Non- | Conforming | Non- |
| (Positive | | | | | irritating | | conforming | | conforming |
| control) | | | | | | | | | |

TABLE 24
Body wash

| | Sum of skin | | | | | | | Irritation of | | |
|---|---|---|---|---|---|---|---|---|---|---|
| | irritation | | | | | | Irritation | level ++ or | | |
| | reaction | | | Skin | Skin | | in >20% | more in >10% | | |
| Sample | intensity | | | irritation | irritation | Safety | of test | of test | | |
| name | 1^{st} | 2^{nd} | 3^{rd} | index | rating | zone | subjects | subjects | Conformity | Comment |
| Cream of | 0.00 | 0.00 | 0.00 | 0.00 | Not | Conforming | Conforming | Conforming | Conforming | 1% |
| present | | | | | irritating | | | | | Dilution |
| invention | | | | | | | | | | |
| D.W. | 0.00 | 0.00 | 0.00 | 0.00 | Not | Conforming | Conforming | Conforming | Conforming | — |
| (Negative | | | | | irritating | | | | | |
| control) | | | | | | | | | | |
| 1% SLS | 4.00 | 4.00 | 3.06 | 3.06 | Moderately | Conforming | Non- | Conforming | Non- | — |
| (Positive | | | | | irritating | | conforming | | conforming | |
| control) | | | | | | | | | | |

TABLE 25
Foam cleanser

| | Sum of skin | | | | | | | Irritation of | | |
|---|---|---|---|---|---|---|---|---|---|---|
| | irritation | | | | | | Irritation | level ++ or | | |
| | reaction | | | Skin | Skin | | in >20% | more in >10% | | |
| Sample | intensity | | | irritation | irritation | Safety | of test | of test | | |
| name | 1^{st} | 2^{nd} | 3^{rd} | index | rating | zone | subjects | subjects | Conformity | Comment |
| Foam | 0.00 | 0.00 | 0.00 | 0.00 | Not | Conforming | Conforming | Conforming | Conforming | 1% |
| cleanser of | | | | | irritating | | | | | Dilution |
| present | | | | | | | | | | |
| invention | | | | | | | | | | |
| D.W. | 0.00 | 0.00 | 0.00 | 0.00 | Not | Conforming | Conforming | Conforming | Conforming | — |
| (Negative | | | | | irritating | | | | | |
| control) | | | | | | | | | | |
| 1% SLS | 4.00 | 4.00 | 3.06 | 3.06 | Moderately | Conforming | Non- | Conforming | Non- | — |
| (Positive | | | | | irritating | | conforming | | conforming | |
| control) | | | | | | | | | | |

According to the results shown in Tables 21 to 25, all of the soothing gel, lotion, cream, body wash, and foam cleanser formulations including a green barley extract and ceramide were found to conform to the three final evaluation criteria and were confirmed to be non-irritating products less likely to cause irritation on sensitive skin.

### Experimental Example 8. Human Application Test Results According to Types of Formulation for Evaluating Dryness-Induced Itching Relief Effect of Green Barley Extract and Ceramide

In order to evaluate the dryness-induced itching relief effect of the soothing gel, lotion, and cream formulations including a composition including the ceramide and green barley extract of the present invention, the inventors requested the OATC Skin Clinical Trials Center to perform a human application test.

The degree of dryness-induced itching was subjectively evaluated, before and after four weeks of use of a test product, using a 10-point scale. The evaluation scores are as follows: 1 to 3=the subject scratches unconsciously (itching does not interfere with daily activities or sleep); 4 to 6=the skin is itchy to the extent of interfering with daily activities and sleep (although not all day); 7 to 9=the skin is itchy to the extent of interfering with daily activities and sleep most of the time; 10=the skin is itchy to the extent of causing severe disturbances in daily activities and sleep. The ingredients of the soothing gel, lotion, and cream formulations for the human application test are the same as those described in Tables 8 to 10.

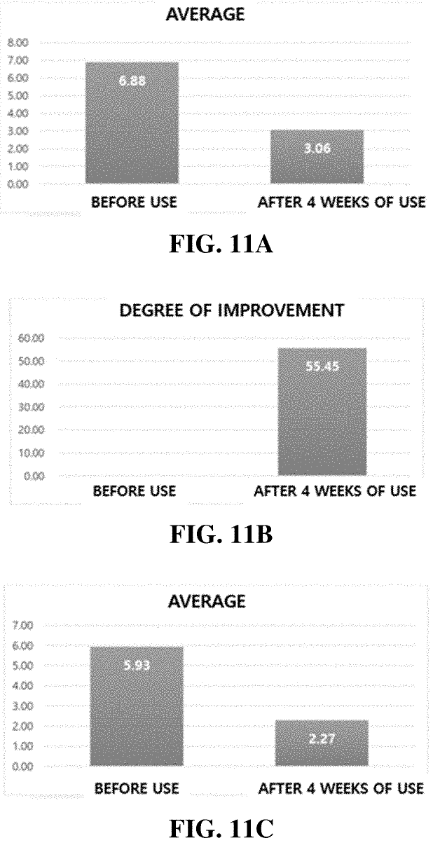

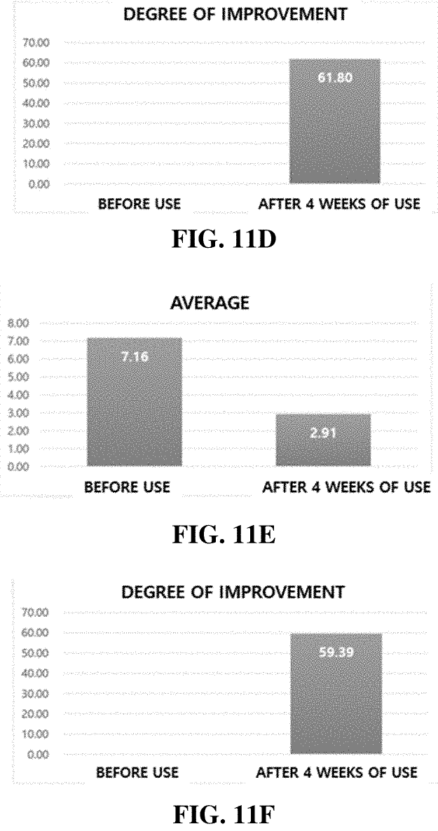

According to the result as shown in FIGS. 11A-11F, in the case of all of the soothing gel, lotion, and cream formulations, the degree of improvement in itching increased after four weeks of use of the formulations. This result suggests that products having the soothing gel, lotion, and cream formulation including the green barley extract and ceramide of the present invention are capable of significantly improving skin dryness-induced itching by moisturizing the skin.

The composition of the present invention, which includes a green barley extract, has excellent skin barrier strengthening and skin moisturizing ability and is a product having excellent safety with a low possibility of causing irritation even on sensitive skin, and thus has high utilization as a cosmetic composition in various formulations and applications.

However, the effects of the present invention are not limited to those described above, and it should be understood that the effects of the present invention include all effects that can be deduced from the configuration of the present invention described in the detailed description or claims of the present invention.

The above description is merely illustrative of the present invention, and a person skilled in the art to which the present invention pertains may understand that the present invention can be easily modified into other specific forms without changing the technical spirit or essential features of the present invention. Therefore, the above-described exemplary embodiments are illustrative and not restrictive in every aspect. For example, components described as an integrated entity may be implemented in a distributed manner, and similarly, components described as being distributed may be implemented in a combined form.

The scope of the present invention is defined by the following claims, and all modifications or variations derived from the meaning and scope of the claims and their equivalents should be construed as being included in the scope of the present invention.

### DESCRIPTION OF REFERENCE NUMERALS

10: TRAY
20: CULTIVATION PLATE
30: STACKING RACK
40: CULTIVATION SOLUTION CIRCULATOR
50: WATER TANK
60: FIRST TRANSFER PIPE
70: SECOND TRANSFER PIPE
100: SPROUT CULTIVATOR

What is claimed is:

1. A cosmetic composition for skin barrier strengthening or skin moisturization comprising a green barley extract, wherein the green barley extract contains saponarin.

2. The cosmetic composition of claim 1, wherein the saponarin is contained at 0.01 to 10% by weight.

3. The cosmetic composition of claim 1, which increases the expression levels of hyaluronic acid, aquaporin 3, and filaggrin.

4. The cosmetic composition of claim 1, wherein the green barley extract is extracted for one to four hours at a temperature of 50 to 70° C. using a 40 to 60% ethanol after being dried at a temperature of 40 to 70° C.

5. The cosmetic composition of claim 1, wherein the green barley extract is included in an amount of 0.001 to 30% by weight based on the total weight of the composition.

6. The cosmetic composition of claim 1, further comprising one or more ceramides selected from the group consisting of ceramide EOP, ceramide NS, ceramide NP, ceramide AS, and ceramide AP.

7. The cosmetic composition of claim 6, wherein the ceramide is included in an amount of 0.01 to 10% by weight based on the total weight of the composition.

8. The cosmetic composition of claim 1, which is in any one of soothing gel, lotion, cream, body wash, foam cleanser, essence, powder, pack, mask, and toner formulations.

9. The cosmetic composition of claim 8, which is also usable for improving itching caused by skin dryness and improving sensitive skin.
